(19) United States
(12) Patent Application Publication
(10) Pub. No.: US 2014/0052033 A1
(43) Pub. Date: Feb. 20, 2014

(54) DEVICE AND METHOD FOR PULSED ACOUSTICAL STIMULATION OF THE BRAIN

(71) Applicants: G. Frank Lawlis, City of Sanger, TX (US); T. Frank Lawlis, City of Celina, TX (US)

(72) Inventors: G. Frank Lawlis, City of Sanger, TX (US); T. Frank Lawlis, City of Celina, TX (US)

(73) Assignee: BAUD Energetics, Corp, Celina, TX (US)

(21) Appl. No.: 13/589,505

(22) Filed: Aug. 20, 2012

(51) Int. Cl. A61N 1/00

(52) U.S. Cl. 601/47

(57) ABSTRACT

An electronic device for stimulating the brain of a living subject. The device comprises a signal generator configured to generate an acoustic signal that the subject can sense. The device also comprises a user interface coupled to the signal generator and configured to allow the subject to change the acoustic signal by pulsing the acoustic signal on and off at a pulse rate, to thereby stimulate the subject's brain into a targeted state of activity.

### TECHNICAL FIELD

This application is directed, in general, to electronic devices and, more specifically, to an electronic device for generating acoustic signal to stimulate the brain of a living subject and methods of using such a device.

### BACKGROUND

Brain activity can be affected by acoustic stimulation. Some methods for acoustically stimulating the brain, e.g., to improve health, rely on creating a tertiary signal (sometimes known as a binaural signal) as the difference between two constant acoustic signals having different frequencies (also known as tone or pitch).

### SUMMARY

One embodiment is an electronic device for stimulating the brain of a living subject. The device comprises a signal generator configured to generate an acoustic signal that the subject can sense. The device also comprises a user interface coupled to the signal generator and configured to allow the subject to change the acoustic signal by pulsing the acoustic signal on and off at a pulse rate, to thereby stimulate the subject's brain into a targeted state of activity.

Another embodiment of the disclosure is a method of stimulating the brain of a living subject. The method comprises generating, using an electronic device, an acoustic signal that the subject can sense. The method also comprises adjusting the acoustic signal, using the electronic device, to find a resonant acoustic signal which the subject associates with a first brain activity state of the subject. The method further comprises changing the resonant acoustic signal, using the electronic device, based on feedback from the subject, including pulsing the acoustic signal on and off at a pulse rate to thereby stimulate the subject's brain into a second targeted state of activity.

### BRIEF DESCRIPTION

Reference is now made to the following descriptions taken in conjunction with the accompanying drawings, in which:

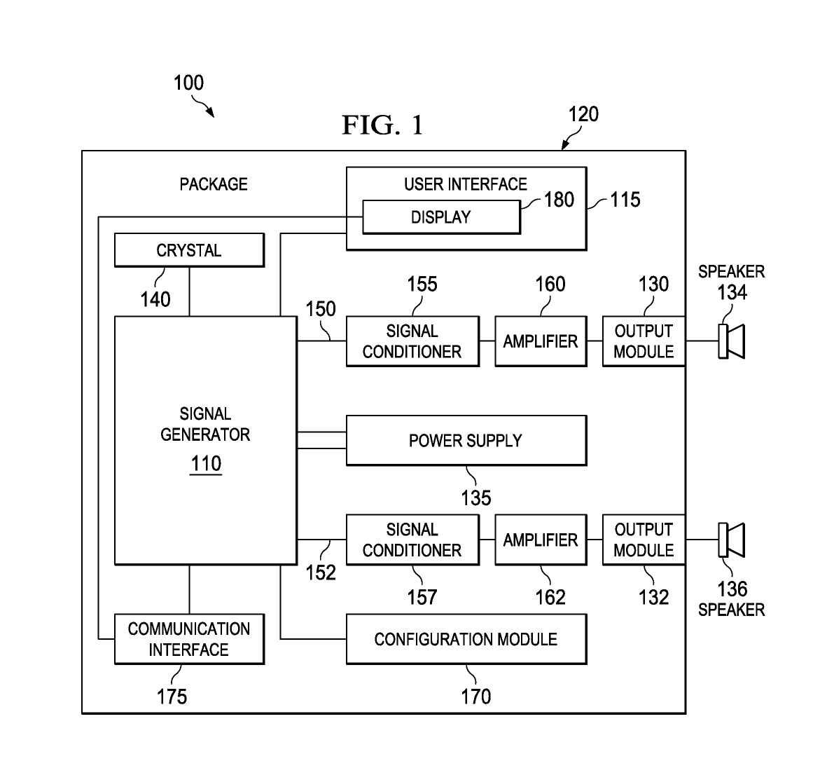

FIG. 1 presents a block diagram of an example electronic device of the disclosure;

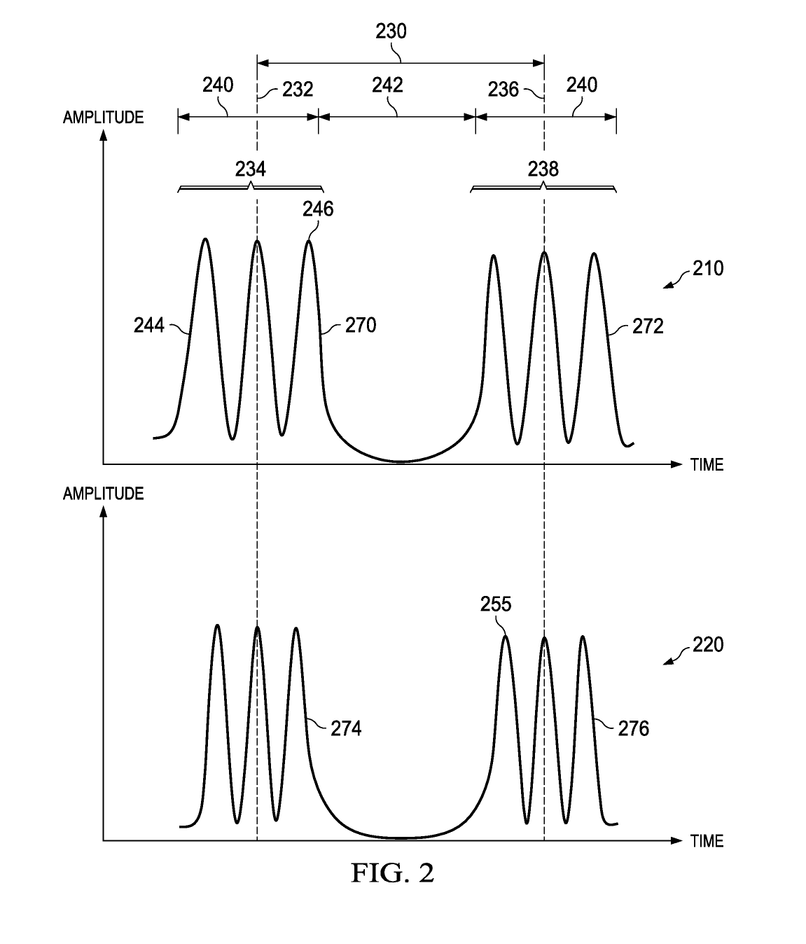

FIG. 2 presents example amplitude versus time profiles for acoustic signals generated in accordance with the principles of the present disclosure, such as acoustic signals generated using the example electronic device illustrated in, and discussed in the context of, FIG. 1;

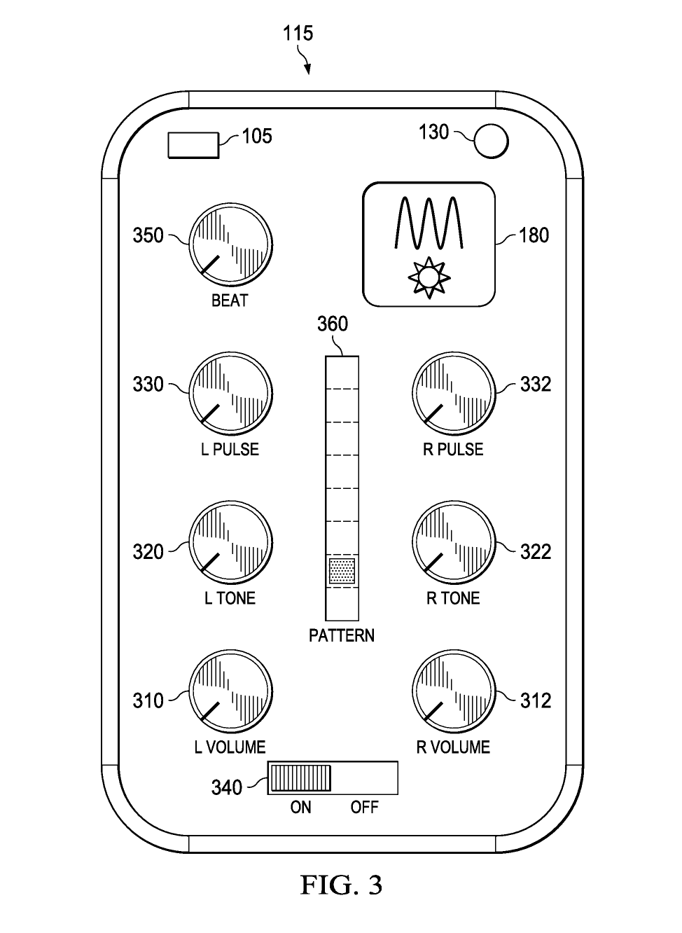

FIG. 3 illustrates an example user interface of the electronic device of the disclosure, such as a user interface of the example device discussed in the context of FIG. 1;

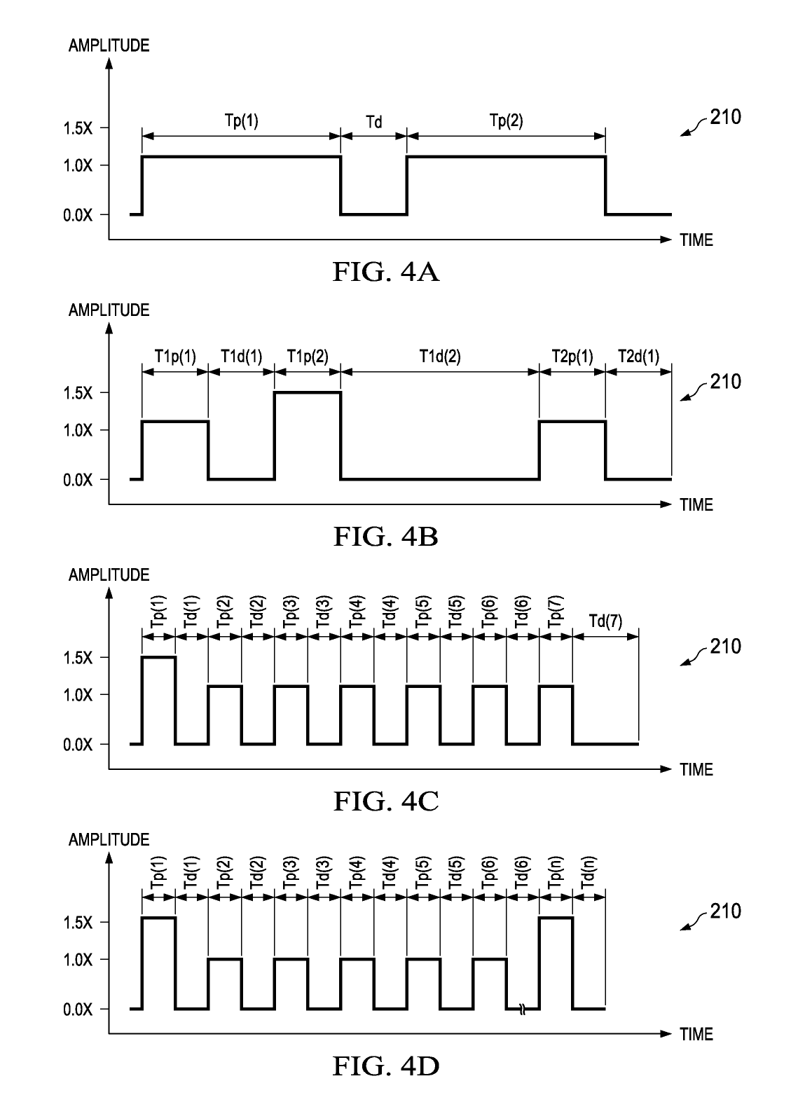

FIG. 4 presents example generated acoustic signals of the disclosure having rhythmic pattern of waveforms; and

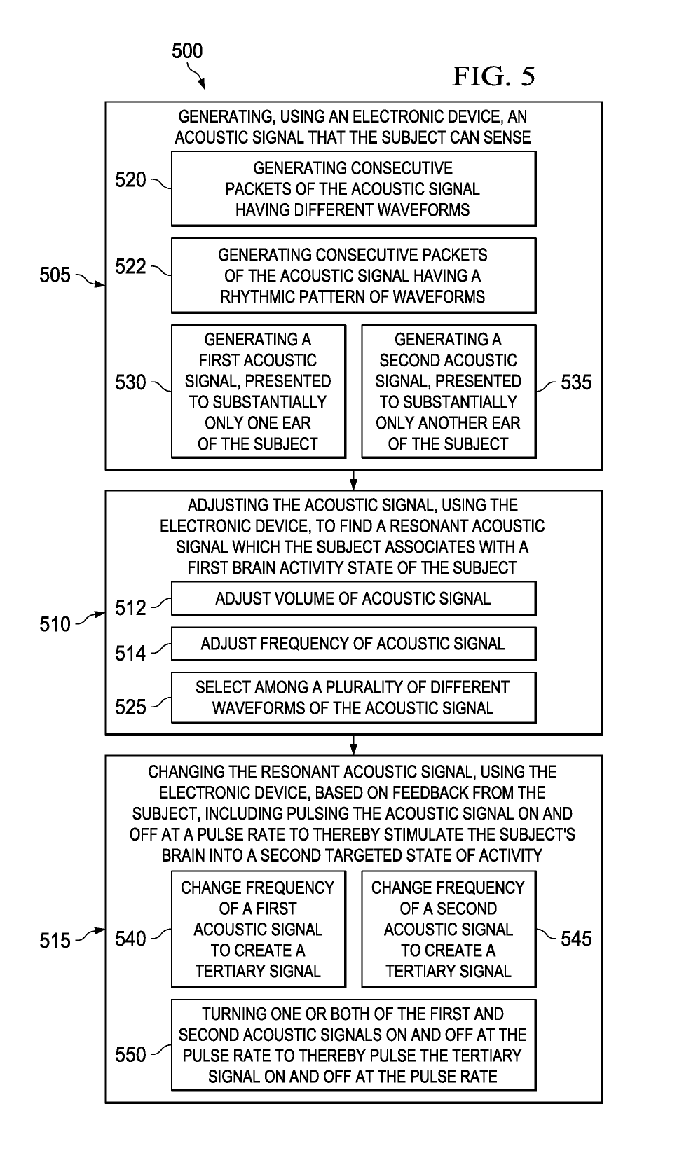

FIG. 5 presents a flow diagram of an example method of stimulating the brain of a living subject, such as implemented using any of the devices, acoustic signals and user interfaces illustrated in, and discussed in the context of, FIGS. 1-4.

### DETAILED DESCRIPTION

The term, “or,” as used herein, refers to a non-exclusive or, unless otherwise indicated. Also, the various embodiments described herein are not necessarily mutually exclusive, as some embodiments can be combined with one or more other embodiments to form new embodiments.

It was discovered, as part of the present disclosure, that acoustically stimulating the brain, e.g., to reach particular target brain activity state, is improved by providing subjects with the ability (e.g., through an electronic device having a user interface) to adjust the pulse rate of an acoustic signal. This in contrast with some previous efforts where subjects were provided with the ability to adjust the beat of a tertiary signal by adjusting one or both of the frequencies of the two constant acoustic signals presented to different ears of the subject, as disclosed in U.S. Pat. Nos. 7,166,070, and 7,354,395, both to Lawlis et al., and both of which are incorporated by reference as if reproduced in their entirety herein.

The efficacy of providing a subject with the ability to adjust the acoustic signal's pulse rate, as disclosed herein, was surprising because it was thought that effective brain stimulation required the production and adjustment of the tertiary signal's beat. In some cases, the effective stimulation of the brain, by providing the subject with the ability to adjust a pulse rate of the acoustic signal's pulse rate, can have a number of technical advantages as compared to providing and adjusting a tertiary signal's beat. For instance, it is not necessary to generate, in an electronic device, two constant acoustic signals in each ear and two signals with different frequencies, respectively, to thereby produce a constant tertiary signal. Consequently, the electronic device does not have to have two earpieces or even two speakers to generate a single-frequency acoustic signal.

For instance, it is not necessary for the electronic device to hold one of the constant acoustic signals at one frequency while changing the frequency of the constant second acoustic signal to different frequency to thereby create or adjust the beat of the tertiary signal, or, for the electronic device to change both the first and second constant acoustic signals to two different constant acoustic signals to thereby create or adjust the beat of the tertiary signal. Consequently, the electronic device does not have to have as complex or precise a signal generator, signal conditioner or amplifier to generate such first, second and tertiary signals. The electronic device merely needs to be able to pulse the acoustic signal on and off, and, to provide a user interface that allows adjustment of the pulse rate of the acoustic signal.

Additional advantageous features of the present disclosure will become apparent in the context of the example embodiments described below.

One embodiment of the disclosure is an electronic device for stimulating the brain of a living subject.

FIG. 1 presents a block diagram of an example electronic device 100 of the disclosure. FIG. 2 presents example amplitude versus time profiles for acoustic signals generated and adjusted in accordance with the principles of the present disclosure, such as acoustic signals 210, 220 generated and adjusted using the example electronic device 100 illustrated in, and discussed in the context of, FIG. 1.

With continuing reference to FIGS. 1 and 2 throughout, the device 100 comprises a signal generator 110 configured to generate an acoustic signal (e.g., acoustic signal 210) that the subject (e.g., humans, dogs, horse, cats, or other mammals, or other organisms having a brain) can sense. The device 100 also comprises a user interface 115 coupled to the signal generator 110 and is configured to allow the subject to change the acoustic signal 210 by pulsing the acoustic signal 210 on and off at a pulse rate 230, to thereby stimulate the living subject's brain into a desired state of activity.

Providing an acoustic signal 210 that the subject can sense, advantageously provides feedback which helps the subject adjust the device 100 (e.g., via the signal generator 110) to find a resonant acoustic signal 210 which the subject associates with a first brain activity state of the subject. In some cases, the acoustic signal 210 can be in an audible frequency range that is sensed by the subject's hearing. For instance, in some cases, the acoustic signal 210 can be a frequency in a range from about 0.1 Hz to about 150,000 Hz, and in some cases, advantageously for human subjects, in a range from about 30 Hz to about 650 Hz. In other cases, the acoustic signal 210 can be a frequency in a range that the subject senses, as a physical vibration, by touch.

In some cases first brain activity state can be a problematic brain activity state in need of therapeutic treatment. The term problematic brain activity state refers to undesired emotions or feelings experienced by the subject in association with a illness, such as a disease or malfunction of the brain or body of the subject. Non-limiting examples include uncontrollable cravings associated with the subject's anticipated future ingestion of an addictive substance, or food, or, of engaging in an addictive activity; pain associated with a particular physical condition of the subject; or depression associated with a real or imagined experience recalled by the subject. Other potential problematic brain activity states would be apparent to one skilled in the art in view of the present disclosure and the incorporated U.S. Pat. Nos. 7,166,070, and 7,354,395 patents.

In other cases, the first brain activity state can be a normal healthy brain activity state, e.g., due to situation or condition experienced by the subject, but a state that the subject wishes to better control, or, in some cases, enhance, and, which is not in need of therapeutic treatment. Non-limiting examples include: controlling feelings of anxiety associated with the subject's preparation for an exam or a public speech, increased feelings self-confidence or well-being, or increased feelings of weight wellness, associated with decreasing the desire to consume food excessively, or increasing mental focus or having increased attention maintenance. Other potential normal health brain activity states would be apparent to one skilled in the art in view of the present disclosure and the incorporated U.S. Pat. Nos. 7,166,070, and 7,354,395 patents.

In some embodiments, the device 100 has a therapeutic use and is used in a therapeutic method. In other embodiments, the device 100 is used in a solely cosmetic method. A person skilled in this art would be able to identify which methods of the invention are therapeutic and which methods are non-therapeutic or cosmetic. For example, when the device is used to reduce feelings of cravings associated with the subject's future ingestion of food in a healthy individual, the method is cosmetic. However, if the device is used to reduce feelings of cravings associated with the subject's future ingestion of food in an obese individual then the method may be regarded as therapeutic. A skilled person would be able to define the different subject groups, for example by assessment of the body mass index (BMI) of the individual.

As illustrated in FIG. 2, by virtue of pulsing the acoustic signal 210 on and off at the pulse rate 230, discrete packets 234, 238 of the generated acoustic signal 210 are formed. The term pulse rate 230 as used herein, refers to the repeating time period between the mid-point 232 of one packet 234 of the acoustic signal 210 and the mid-point 236 of the next packet 238 of the acoustic signal 210. The repeating packets 234, 238 of the acoustic signal 210 are on for an on-cycle 240 and are separated by an off cycle 242. The acoustic signal 210 is defined as being on, if the amplitude 244 of a packet 234 at a give time is equal to or greater than about 10 percent of the peak amplitude 246 of the packet 234, and being off, if the amplitude 244 is less than about 10 percent of the peak amplitude 246. The acoustic signal 210 has on-cycles 240 that are sufficiently long for the subject to sense, e.g., to consciously perceive the acoustic signal 210 when it is on. For instance, in some cases for a human subject and when using an acoustic signal 210 in the audible hearing range, each of the on-cycles 240 is at least about 100 ms and in some cases, at least about 1000 ms, and in still other cases, at least about 5000 ms in length. In some embodiments the pulse rate 230 is at a frequency in a range from about 0.1 to 25 Hz, and in some cases, a frequency in a range from about 4 Hz to about 7 Hz. The latter range has been found to be advantageous for stimulating certain target brain activities, which may include producing the theta waveform of brain wave activity in human subjects

The example device 100 shown in FIG. 1 can include a package 120 and sound output modules 130 and 132 (e.g., one or more ports for connection to earpieces 134, 136 for the left and right ear or for connection to stand-alone speakers 134, 136 situated near the left and right ears, respectively). The package 120 can hold the user interface 115, the microprocessor-based signal generator 110 and a power supply 135. Some embodiments of the signal generator 110 can be analog circuits or integrated circuits, such as a Microchip Technology, Inc. PIC18F452 micro controller. The signal generator 110 can have an associated crystal 140 for timing the microprocessor, which in some cases, can be a 9.8304 MHz crystal. The signal generator 110 can be configured to output at least one, and in some cases two, pulse code modulated (PCM) wave forms (e.g., sine wave forms 270, 272, 274, 276), which can serve as input 150, 152 to one or two signal conditioners 155, 157, respectively. The signal conditioner 155, or in some cases, conditioners 155, 157, are configured, using procedures familiar to those skilled in the art, to condition the waves for amplification by one amplifier 160, and in some cases two amplifiers 160, 162, respectively. The amplified signals can serve as input to the sound device 130 or output modules 130, 132.

In some embodiments, as illustrated in FIG. 1 the device 100 includes a configuration module 170 (e.g., jumper pins or dip switches) for providing functional control over the device 100. In some embodiments, as illustrated in FIG. 1 the device 100 includes a communication interface 175 (e.g., an RS-232 or USB interface) for communicating with the signal generator 110, such as for downloading programs of waveforms 270, 272, 274, 276 into a memory of the signal generator 110, or, for receiving signals corresponding to present brain wave activity or systemic physiological signs from the subject for presentation to the user interface 115. Such signals can be presented on a display 180 of the user interface 115 to facilitate the subject achieving the targeted state of brain activity.

FIG. 3 illustrates an example user interface 115 of the electronic device of the disclosure, such as the example device 100 illustrated in, and discussed in the context of, FIG. 1. As illustrated in FIG. 3, the user interface 115 can have user-adjustable control features (e.g., rotationally adjustable knobs) to control the volume 310, frequency (as referred to as pitch or tone) 320 and pulse rate 330 of the acoustic signal 210. Some embodiments of the user interface 115 can further include an on/off switch 340 (e.g., a slide switch), the sound output module 130 (e.g., mini-jack, ¼-inch or 2.5 mm port styles), and the communication interface 175.

As further illustrated in FIG. 3, some embodiments of the user interface 115 provide control features for independent control of the acoustic signal presented to the left (L) and right (R) ears of the subject, e.g., left and right volume 310, 312, frequency (e.g., pitch or tone) 320, 322, and pulse rate 330, 332 controls.

In some cases, brain stimulation using the device or methods described herein, can be performed without regard to the then present brain wave activity or systemic (e.g., non-brain) physiological signs of the subject.

In other cases, however, indications of one or both of present brain wave activity or systemic (e.g., non-brain) physiological signs can be presented to the subject, to help find the resonant acoustic signal which the subject associates with a first brain activity state (e.g., a problematic state, or, in some cases a normal healthy state). For instance, the electronic device 100 can be further configured to receive signals (e.g., at the interface 175) corresponding to present brain wave activity of the subject, and, the user interface 115 can be configured to display, e.g., on display 350, an indicator of the present brain wave activity. For instance, the electronic device 100 can be further configured to receive signals (e.g., at the interface 175) corresponding to present systemic physiological signs of the subject, and, the user interface 115 can be configured to display, e.g., on display 180, an indicator of the present systemic physiological signs. One skilled in the art would be familiar with methods and devices to measure brain wave activity (e.g., electroencephalography or similar devices) and how to convert such measurements into electronic signals to be transferred to the device 100. Similarly, one skilled in the art would be familiar with methods and devices to measure systemic physiological sign, such as Galvanic skin response; heart rate; blood pressure; respiration rate, and, such measurements into electronic signals to be transferred to the device 100. The display 180 can represent such indications as numbers, wave forms, progress bars or meters, colored lights flashing lights, or other representations familiar to those skilled in the art.

As discussed above, some embodiments of the device and method disclosed herein present a single acoustic signal 210 (e.g., a same frequency acoustic signal to one or both ears) and the subject can adjust the pulse rate of the acoustic signal to thereby stimulate the subject's brain into the targeted state of activity.

In other embodiments, however, the acoustic signal includes a first acoustic signal 210 of one frequency presented to substantially only one ear of the subject. A second acoustic signal 220 of another different frequency is presented to substantially only a second ear of the subject, and, the frequency differential between the first and second acoustic signals 210, 220 (e.g., the sum of the respective waveforms 270, 272, 274, 276 of the two acoustic signals 210, 220) creates a tertiary signal having a beat frequency, sometimes referred to as a binaural beat frequency. The user interface 115 is further configured to allow the subject to independently change (e.g., via separate adjustment knobs 320, 322) the frequencies of the first and second acoustic signals 210, 220 and thereby the beat frequency (e.g., via adjustment knob 350) of the tertiary signal. Such embodiments thereby provide the subject with a supplementary means, in addition to changing the pulse rate of the acoustic signal, to stimulate the subject's brain into the targeted state of activity. In some cases, the beat frequency can be in a range of about 0.1 to about 20 Hz, and in some cases about 1 to about 7 Hz.

In some such embodiments, it is advantageous, as part of stimulating the subject's brain, to further allow the subject to change the pulse rates of one or both of the first and second acoustic signals 210, 220, to thereby pulse the tertiary signal on and off at the pulse rate. For example, in some cases, the first acoustic signal 210 and/or the second acoustic signal 220 turned on and off at the pulse rate to pulse the tertiary signal on and off at the pulse rate 230. In some cases the first and second acoustic signals 210, 220 are both turned on and off at the pulse rate 230 substantially simultaneously (e.g., within about 100 ms of each other in some cases, and within about 10 ms in other cases, and within about 1 ms in other cases). Turning the signals 210, 220 off and on substantially simultaneously can help to ensure that the consecutive waveforms 270, 272 of the first acoustic signal 210 are on at substantially the same time as the consecutive waveforms 274, 276 of the second acoustic signal 220.

Presenting the first acoustic signal 210 of one frequency presented to substantially only to one ear and the second acoustic signal 220 of another different frequency to presented substantially only to a second ear of the subject helps avoid the formation of acoustic interference between the two acoustic signals 210, 220. The term the first acoustic signal 210 presented to substantially only one ear, as used herein, means that the sound level of the second acoustic signal 220 reaching the first ear that directly receives the first acoustic signal 210 is less than 10 percent of the sound level of the first acoustic signal at that first ear. Likewise, the term the second acoustic signal 220 presented to substantially the second ear, as used herein, means that the sound level of the first acoustic signal 210 reaching the second ear that directly receives the second acoustic signal 210 is less than about 10 percent of the sound level of the second acoustic signal 220 at the second ear.

In some embodiments, to improve stimulating the subjects brain into a targeted state of activity, the signal generator 110 can be programmed to generate a waveform 270 of the acoustic signal 210, or in some cases, waveforms 270, 274 of the first and second signals 210, 220, that have a constant pattern such as sine functions with constant amplitudes, as illustrated in FIG. 2. In other embodiments, however, to enhance the stimulation of the subject's brain into the targeted state of activity the generated waveform 270, or waveforms 270, 274 can have more complex patterns. In some cases, for example, the generated waveform 270, or waveforms 270, 274 can be sine function having a non-uniform amplitude. In other cases, for example, the generated waveform 270, or waveforms 270, 274 can be a triangular, square or saw-tooth waveforms, or other type of waveform function, or combinations thereof. In some embodiments of the device 100, the user interface 115 can be configured to include an adjustment switch 360 (e.g., a multi-position slide switch), to allow the subject to select among several different patterns of waveforms, e.g., that are designed to facilitate the subject finding a resonant acoustic signal which the subject associates with various different first brain activity states.

In some embodiments, to improve stimulating the subject's brain into a targeted state of activity the waveforms 270, 272 of the packets 234, 238 of the generated acoustic signal 210 can differ from each other. For instance, the signal generator 110 can be configured to alternate the waveforms 270, 272 of the consecutive packets 234, 238 being generated between two or more different waveforms. For instance, a first waveform 270, defined by a sine function with a constant peak amplitude 246, can be alternated with a second waveform 272, defined by a sine function with a gradually increasing peak amplitude 246. For instance, a first waveform 270, defined by a sine function with a constant peak amplitude 246, may be alternated with a second waveform 272, defined by a saw-tooth function with a constant peak amplitude 246. Similarly, in still other embodiments, the consecutive waveforms 270, 272 of the first acoustic signal 210 can differ from the consecutive waveforms 274, 276 of the second acoustic signal 220.

Based on the present disclosure, one skilled in the art would appreciate how other more complex patterns of the acoustic signal 210, or signals 210, 220, could be produced by the signal generator 110 to facilitate association with various brain activity states.

For example, FIG. 4 presents example generated acoustic signals 210 of the disclosure having a rhythmic pattern of waveforms 270. That is, the generated waveforms 270 can have complex patterns which form a rhythmic pattern. For instance, the consecutive packets 234, 238 can include or be repeating rhythmic patterns of waveforms. Such rhythmic waveform patterns have been discovered to be particularly useful at facilitating a subject's ability to efficiently find the resonant acoustic signal associated with various specific brain activity states. In some embodiments, the device 100 is configured to allow the subject to select among such generated waveforms 270, and in some cases, to customize these rhythmic waveform patterns.

In these examples, a square-wave waveform function is used for illustrative purpose to show various waveforms 270. However, any of the above-described waveform functions, or other waveforms familiar to those skilled in the art, could be used. The characteristics of generated example waveform patterns are described using the following terms, with reference to FIG. 4. The acoustic signal is pulsed on (e.g., at some arbitrary scale non-zero amplitude, 1.0× or 1.5×) for some period of time (e.g., an acoustic on-cycle pulse time 240, Tp) and off, (e.g., substantially zero amplitude, 0.0× on the arbitrary scale) for some period of time (e.g., an off-cycle 242 inter-pulse delay time, Td). In the case of the example rhythmic waveform 270 patterns, a “meter” refers to the number of acoustic pulses per one complete cycle of the repeating pattern. The repeating pattern of pulses (i.e., the pulse rate), is repeated at a frequency defined as a “tempo.” The desired tempo and a duty cycle (e.g., of the signal generator 110) defines how long each acoustic pulse is “on” (Tp) and the length of the inter-pulse delay time (Td), during each meter of the repeating pattern.

A repeating rhythmic pattern of a steady-amplitude pulsed acoustic signal 210, is illustrated in FIG. 4(A). Some such embodiments can be configured to approximate a theta waveform. Such a generated waveform has been found useful for inducing a brain activity state that include one or more of deep relaxation, dream-like sensation and imagery, or heighten suggestibility or hypnotic states. In some cases, e.g., the pulse rate, or tempo, of such as pattern can equal about 7 Hz. and the signal generator (e.g., generator 110, FIG. 1) has a duty cycle of about 40 percent. For such an example embodiment, the pulse rate 230 equals about 142 ms, the length of the on-cycle 240 equals about 57 ms (Tp=57 ms), and each of the pluses are separated by an off-cycle 242 or inter-pulse delay time of about 86 ms (Td=86 ms). In other cases, the same type of repeating pattern constant amplitude acoustic signals can have a pulse rate, or tempo, of 4 Hz and a duty cycle of about 40 percent. The pulse rate 230 equals about 250 ms, the length of the on-cycle 240 equals about 100 ms (Tp=100 ms) and each of the pulses are separated by an off-cycle 242 or inter-pulse delay time of about 150 ms (Td=150 ms). As illustrated, in some cases, as illustrated in FIG. 4(A), all of the acoustic signals are on for the same duration (e.g., the length of the first on-cycle 240, Tp(1), equals the length of the next on-cycle 240 Tp(2) during a meter). In other cases, however, the acoustic signals can be on for different durations (e.g., the length of the first on-cycle 240, Tp(1), does not equal the length of the next on-cycle 240, Tp(2) during a meter), e.g., by varying the duty cycle of the generator 110 during a meter.

An example repeating rhythmic pattern of a “heartbeat” pattern of the acoustic signal 210, is illustrated in FIG. 4(B). Such a generated waveform has been found useful for inducing a brain activity state that is associated with deep sleep and sense of security. In the example embodiment, the meter has two non-zero amplitude acoustic pulses: a first pulse (e.g., of duration T1p(1) having a non-zero amplitude 1.0×, and a second pulse (e.g., of duration T1p(2) having a non zero amplitude 1.5×, separated by an inter-pulse delay time T1d(1). In some cases, a second inter-pulse delay time (e.g., T1d(2) between the second pulse (e.g., T1p(2) and the first pulse of the next meter can be at least as long as twice the first inter-pulse delay time plus a constant pulse length first and second pulses (e.g., Tp(1), Tp(2) are equal, and, T2(d)≧2T1(d)+Tp(1)). Thus, the second inter-pulse delay time can be thought of as including a third zero-amplitude pulse, or “silent” pulse, in between the second pulse of the first meter and the first pulse of the second meter. Similar to that discussed in the context of FIG. 4(A), in some cases such as illustrated in FIG. 4(B), all of the acoustic signals can be on for the same duration. For example, the length of the first on-cycle 240, T1p(1), is equal to the length of the next on-cycle 240 T1p(2), during the meter. In other cases, however, the acoustic signals can be on for different durations (e.g., the length of the first on-cycle 240, T1p(1), does not equal the length of the next on-cycle 240, T1p(2) during a meter), e.g., by varying the duty cycle of the generator 110 during the meter.

An example repeating rhythmic pattern of a “march” pattern of the acoustic signal 210, is illustrated in FIG. 4(C). Such a generated waveform has been found useful for inducing a brain activity state that is associated with or more of high aspirations and goals, or a sense of union with others in goals. In the example embodiment, the meter has seven non-zero amplitude acoustic pulses, Tp(1) . . . Tp(7) each being separated by seven inter-pulse delay times, Td(1) . . . Td(7), respectively. As illustrated, in some cases, the first pulse can have an amplitude that is 1.5 times larger (e.g., 1.5×) than the amplitude of the following six pulses (e.g., 1×). As illustrated, the first six inter pulse delay times (Td(1) . . . Td(6)) can have equal durations, and the seventh inter pulse delay time (Td(7)) can be at least as long as twice any one of the first six inter-pulse delay time plus a constant pulse length the pulses (e.g., Tp(1) . . . Tp(7) are equal, and, Td(7)≧2T1(d)+Tp(1)). Thus, the seventh inter-pulse delay time can be thought of as including an eighth zero-amplitude pulse, or silent pulse, in between the seventh pulse of the first meter and the first pulse of the second meter.

An example customizable rhythmic pattern of the acoustic signal 210, is illustrated in FIG. 4(D). The generated waveform can be defined by the subject, to facilitate associating the signal 210 with a particular brain activity state. In the example embodiment, the meter has n non-zero amplitude acoustic pulses, Tp(1) . . . Tp(n) each pulse being separated by n inter-pulse delay times, Td(1) . . . Td(n), respectively, where n is between 2 and 12 (e.g., n=7 in the example illustrated in FIG. 4(D)). In some cases, the first and last pulse can have amplitudes that are 1.5 times larger (e.g., 1.5×) than the amplitude of the middle pulses (e.g., 1.0×). As illustrated, the pulse delay times (Td(1) . . . Td(7)) can have equal durations. In other cases, the first and last pulse can have zero amplitude, that is, the first and last pulses can be consider to be silent pulses, similar to that discussed above in the context of FIGS. 1(B) and 1(C).

The electronic device 100 described in the context of FIG. 1 is an example embodiment of an application-specific device of the disclosure. Other embodiments the device, however, can be implemented as part of a multi-functional electronic device, such as a smart phone or a personal computer. In some such embodiments, the hardware for the signal generator 110, user interface 115, and the other above-described components, can be provided as part of the multi-functional device, in the form of computer programs that can be down-loaded into the multi-functional device, to thereby provide commands to control the appropriate hardware of the multi-functional device in accordance with the principles and methods disclosed herein. For instance, the signal generator 110 can be implemented as a computer program stored in the memory, and configured to be loaded into the central processing unit, of a multi-functional device to control the speaker output of the multi-functional device. For instance, the user interface 115 can be implemented as another computer program stored in the memory, and configured to present virtual control features (e.g., virtual adjustable knobs or switches), on a display screen of the multi-functional device.

Another embodiment of the disclosure is a method of stimulating the brain of a living subject. FIG. 5 presents a flow diagram of an example method 500 of stimulating the brain of a living subject, such as implemented using any of the devices 100, acoustic signal 210, or signals 210, 220, and user interface 115 illustrated in, and as discussed in the context of, FIGS. 1-4.

With continuing reference to FIGS. 1-4 throughout, the method 500 comprises a step 505 of generating, using an electronic device 100, an acoustic signal 210 that the subject can sense. The method 500 further comprises a step 510 of adjusting the acoustic signal 210, using the electronic device 100, to find a resonant acoustic signal 210 which the subject associates with a first brain activity state of the subject. In some embodiments of the method 500, adjusting the acoustic signal 210 in step 510 includes adjusting at least one, and in some cases both, of a frequency (step 512) or volume (step 514) of the acoustic signal (e.g., using adjustment knobs 310, 320) as part of finding the resonant acoustic signal. The method 500 also comprises a step 515 of changing the resonant acoustic signal 210, using the electronic device 100, based on feedback from the subject, including pulsing the acoustic signal 210 on and off at a pulse rate 230 to thereby stimulate the subject's brain into a targeted state of activity.

In some embodiments, changing the acoustic signal in step 515 disrupts the first brain activity of the subject, for instance, as evaluated in a survey or questionnaire filled out by the subject. In some cases, e.g., a first brain activity state, that includes feelings of anxiety associated with the subject's preparation for an exam or speech, is disrupted when the acoustic signal 210 is pulsed on and off at a pulse rate, and, the subject's brain is stimulated to a second targeted state of activity of feeling confident associated with preparing for the exam or speech. For instance, the subject's connection between feelings of anxiety associated with preparing for the exam or speech is disrupted, and, a new connection between feelings of confidence associated with preparing for the exam or speech is stimulated.

In some embodiments, the feedback from the subject to find the resonant acoustic signal in step 515 includes indications from the subject's subjective sensations or feelings. In another cases the feedback includes additionally, or alternatively, indications of brain activity measurements of the subject (e.g., the subject's present electricity brain wave activity, as measured using an electroencephalography device) and/or indications of physiological signs of the subject (e.g., a present Galvanic skin response, heart rate, blood pressure, and/or respiration rate).

In some embodiments, changing the resonant acoustic signal in step 515 includes adjusting the pulse rate to a frequency in a range from about 0.1 Hz to about 25 Hz, and in some cases a frequency in a range from about 4 Hz to about 7 Hz (e.g., to correspond to the theta waveform of brain wave activity of human subjects).

In some embodiments, generating the acoustic signal, in step 505, further includes a step 520 of generating consecutive packets 232, 234 of the acoustic signal having different waveforms 270. In some embodiments, generating the acoustic signal, in step 505, further includes a step 522 of generating consecutive packets 232, 234 of the acoustic signal having a rhythmic pattern of waveform, such as any of the patterns discussed in the context of FIG. 4. In some such instances, the subject, as part of the step 510 of adjusting the acoustic signal 210 to find the resonant acoustic signal can select in step 525, using the electronic device 100 (e.g., via a switch 360 on the user interface 115) among a plurality of different waveforms 270 of the acoustic signal 210, including among different rhythmic patterns of waveforms.

In some embodiments generating the acoustic signal 210 in step 505 includes a step 530 of generating a first acoustic signal 210, presented to substantially only one ear of the subject, and, a step 535 of generating a second acoustic signal 220, presented to substantially only a second ear of the subject. In some such embodiments, adjusting the acoustic signal to find the resonant acoustic signal in step 510 can include adjusting the both the first and the second acoustic signals in unison. For example, either both the volume and frequency of the first and the second acoustic signals can be adjusted at the same time and to the same extent as part of steps 512, and 514, respectively, until the resonant acoustic signal which the subject associates with a first brain activity state is found. In some embodiments, changing the resonant acoustic signal in step 515 further includes a step 540 of changing a frequency of the first acoustic signal 210, or, a step 545 of changing a frequency of the second acoustic signal 220, or, a combination of both steps 540 and 545, to create a difference in the frequencies generated by the first and second acoustic signal. The frequency difference between the first and second acoustic signals 210, 220 creates a tertiary signal having a beat frequency which thereby stimulates the subject's brain into the second targeted state of activity.

For example, in some cases, after finding the resonant acoustic signal, the subject may hold the frequency of the first acoustic signal 210 constant and adjust the frequency of the second acoustic signal 220 to a different value to thereby create the tertiary signal having the beat frequency, which then stimulate the subject's brain into the targeted state of activity. In some cases, the beat frequency created by adjusting the frequencies of one or both of the first or second acoustic signals 210, 212 is in a range from about 0.1 Hz to about 20 Hz, and in some cases about 1 Hz to about 7 Hz.

It is believed that, for some embodiments of the method 500, achieving the second targeted state of brain activity can be facilitated by creating tertiary signal with the beat frequency as described about and then pulsing the tertiary signal at the pulse rate. For instance, in some embodiments, changing the resonant acoustic signal in accordance with step 515 includes, in step 550, turning one or both of the first and second acoustic signals 210, 220 on and off at the pulse rate 230 to thereby pulse the tertiary signal on and off at the pulse rate 230. For example, in some embodiments, the tertiary signal is pulsed on and off at a frequency in a range from about 0.1 Hz to about 20 Hz, and in some cases about 4 Hz to about 7 Hz. As discussed above, in some cases the first and second acoustic signals 210, 220 are both turned on and off substantially simultaneously.

Those skilled in the art to which this application relates will appreciate that other and further additions, deletions, substitutions and modifications may be made to the described embodiments.

What is claimed is:

1. An electronic device for stimulating the brain of a living subject, comprising:
a signal generator configured to generate an acoustic signal that the subject can sense; and
a user interface coupled to the signal generator and configured to allow the subject to change the acoustic signal by pulsing the acoustic signal on and off at a pulse rate, to thereby stimulate the subject's brain into a targeted state of activity.

2. The device of claim 1, wherein the electronic device is further configured to permit the subject to adjust one or more of a frequency or volume of the acoustic signal.

3. The device of claim 1, wherein the electronic device is further configured to receive signals corresponding to present brain wave activity of the subject and the user interface is configured to display an indicator of the present brain wave activity.

4. The device of claim 1, wherein the electronic device is further configured to receive signals corresponding to present systemic physiological signs of the subject and the user interface is configured to display an indicator of the present systemic physiological signs.

5. The device of claim 1, wherein the acoustic signal generated by the signal generator includes a first acoustic signal of one frequency presented to substantially only one ear of the subject and a second acoustic signal of another different frequency presented to substantially only a second ear of the subject, wherein the difference between the first and second acoustic signals creates a tertiary signal having a beat frequency; and
wherein the user interface is further configured to allow the subject to independently change the frequencies of the first and second acoustic signals and the beat frequency of the tertiary signal.

6. The device of claim 5, wherein the subject's control of the pulse rate includes changing the pulse rates of the first and second acoustic signals, thereby allowing the subject to pulse the tertiary signal on an off at the pulse rate.

7. The device of claim 6, wherein the first acoustic signal and the second acoustic signal are both turned on and off at the pulse rate substantially simultaneously.

8. The device of claim 1, wherein the signal generator is configured to generate consecutive packets of the acoustic signal which have different waveforms.

9. The device of claim 1, wherein the signal generator is configured to generate consecutive packets of the acoustic signal which include rhythmic patterns of waveforms.

10. The device of claim 1, wherein the signal generator and user interface are implemented as computer programs on a smart phone or personal computer.

11. A method of stimulating the brain of a living subject, comprising:
generating, using an electronic device, an acoustic signal that the subject can sense;
adjusting the acoustic signal, using the electronic device, to find a resonant acoustic signal which the subject associates with a first brain activity state of the subject; and
changing the resonant acoustic signal, using the electronic device, based on feedback from the subject, including pulsing the acoustic signal on and off at a pulse rate to thereby stimulate the subject's brain into a second targeted state of activity.

12. The method of claim 11, wherein the subject is a healthy individual, the first brain activity state is a normal healthy brain activity state and the method is a non-therapeutic method.

13. The method of claim 12, wherein the first brain activity state comprises feelings of cravings associated with the subject's future ingestion of food.

14. The method of claim 11, wherein the subject is ill, the first brain activity state is a problematic brain activity state and the method is a therapeutic method.

15. The method of claim 11, wherein adjusting the acoustic signal includes adjusting at least one of a frequency or volume of the acoustic signal as part of finding the resonant acoustic signal.

16. The method of claim 11, wherein changing the acoustic signal disrupts the first brain activity of the subject.

17. The method of claim 11, wherein the feedback from the subject includes indications of brain activity measurements of the subject.

18. The method of claim 11, wherein the feedback from the subject includes indications of physiological signs of the subject

19. The method of claim 11, wherein changing the resonant acoustic signal includes adjusting the pulse rate to a frequency in a range from about 0.1 Hz to about 25 Hz.

20. The method of claim 11, wherein changing the resonant acoustic signal includes adjusting the pulse rate to a frequency in a range from about 4 Hz to about 7 Hz.

21. The method of claim 11, wherein generating the acoustic signal includes generating consecutive packets of the acoustic signal having different waveforms.

22. The method of claim 11, wherein generating the acoustic signal includes generating consecutive packets of the acoustic signal having a rhythmic pattern of waveforms.

23. The method of claim 11, wherein generating the acoustic signal includes generating a first acoustic signal, presented to substantially only one ear of the subject, and, a second acoustic signal, presented to substantially only a second ear of the subject;
wherein adjusting the acoustic signal to find a resonant acoustic signal includes adjusting the first and second acoustic signals in unison; and
changing the resonant acoustic signal, based on feedback from the subject, further includes changing one or both of the frequencies of the first and second acoustic signals to create a difference in frequencies of generated by the first and second acoustic signal, wherein the frequency difference between the first and second acoustic signals creates a tertiary signal having a beat frequency which thereby stimulate the subject's brain into the second targeted state of activity.

24. The method of claim 23, wherein changing the resonant acoustic signal includes substantially simultaneously turning the first and second acoustic signals on and off at the pulse rate to thereby pulse the tertiary signal on and off at the pulse rate.

25. The method of claim 24, wherein the pulse rate of the tertiary signal is at a frequency in a range from about 0.1 Hz to about 20 Hz.
